(19) United States
(12) Patent Application Publication
(10) Pub. No.: US 2026/0321382 A1
(43) Pub. Date: Oct. 15, 2026

(54) Integrated electronic health record instructional scannable item

(71) Applicant: SHL Medical AG, Zug (CH)

(72) Inventors: James Thomas Hawthorne, Boonton, NJ (US); Christopher James Franzese, Boonton, NJ (US)

(21) Appl. No.: 19/109,344

(22) PCT Filed: Sep. 5, 2023
(86) PCT No.: PCT/EP2023/074335

§ 371 Date: Mar. 6, 2025

Related U.S. Application Data
(60) Provisional application No. 63/405,020, filed on Sep. 9, 2022.

(30) Foreign Application Priority Data
Feb. 16, 2023 (EP) 23157033..4

(51) Int. Cl. G16H 20/13; G16H 10/60

(52) U.S. Cl. CPC G16H 20/13; G16H 10/60

(57) ABSTRACT

The present disclosure provides a method of providing instructions for preparing and verifying the preparation of a medication that is to be administered to a patient using a drug delivery system based upon a medication order. The method includes receiving, at a system, an indication of the medication to be prepared for administration to the patient. The method further includes displaying, on a user interface of the system, a list of one or more items required for medication preparation, including medication, device, and label components. The method further includes scanning, at a scan-enabled client module of the system, a scannable code on each of the one or more items. The method further includes, for at least one item of the one or more items, displaying, on the user interface of the system, a set of one or more instructions specific to the at least one item of the one or more items. The method further includes, after assembling the one or more items required for medication preparation into an assembled unit, verifying one or more features of the assembled unit.

### TECHNICAL FIELD

The present disclosure generally relates to electronic health record systems, and more particularly to an electronic health record system that includes scannable items that provide preparation instructions.

### BACKGROUND

Compounding hoods, such as laminar airflow workbenches (LAFWs), biological safety cabinets (BSCs), and isolators (“glove boxes”) are commonly used for sterile preparation of medications in the health care system. These systems, also known as primary engineering controls (PECs), are typically located within cleanrooms with controlled, high-efficiency particulate absorbing (HEPA)-filtered air to maintain product sterility. Only those materials necessary for medication preparation should be brought into compounding hoods or cleanrooms, and those supplies must be progressively disinfected (e.g., with isopropyl alcohol) prior to introduction. The use of paper materials is strongly discouraged in compounding hoods and cleanrooms due to the potential to release particulate, fiber, and microbes into the air. Personal technologies, such as cell phones, are also prohibited due to the high microbial load carried on such items. Microbes and other particles can contaminate the sterile preparation, which in turn can lead to infection or severe immune response in a patient. These infections can be severe and lead to sepsis and possibly death.

Sterile preparation is conducted by pharmacy technicians, pharmacists, or other healthcare professionals (HCPs) who may prepare hundreds of different medications per day. While many of these preparations are simple and part of routine practice, some require complex preparation steps and manipulations that demand specific instruction. In such cases, medications are typically packaged with preparation instructions, either as designated materials (e.g., pamphlets) or contained within the product's prescribing information. However, as paper is strongly discouraged in the cleanroom, these materials must be discarded before the medication container itself (e.g., vial, syringe) can be brought into the cleanroom and compounding hood. As a result, when items have complicated preparation steps or include devices that are not customary in routine practice, staff are often left to rely on prior training and past experiences to determine how to proceed. Such situations present major risks for preparation errors and demand solutions to ensure steps are completed correctly, especially when they deviate from routine practice.

Some tools already exist to mitigate these risks, although they are not without limitation. In particular, many institutions require their pharmacy staff to use the facility's electronic health record (EHR) in compounding areas to reduce the risk of human error. EHRs are versatile systems that can embed other software or webpages within their interfaces. For example, drug information resources, such as LexiComp, are commonly found embedded in EHR systems for easy user access. Many EHR systems also include software to help guide medication preparation and enforce safety checks by showing the ingredients list or “recipe” for each medication preparation. In such cases, the HCP who is preparing the medication is directed to scan the product identifier (e.g., barcode) for each ingredient of the preparation before they begin compounding. The EHR will then alert the user if the incorrect product is scanned and will ask him/her to scan the correct product prior before they proceed. In this way, the EHR ensures the correct medication ingredients and the correct amounts of those ingredients are being used. For example, if 10 mL of a medication are required and only a 2 mL vial is scanned, the user is directed to scan 5 vials in order to proceed with preparation. These ingredients and the final product may also be subsequently scanned by a second HCP to verify that the medication is prepared correctly.

However, a significant limitation of this tool is that it is designed to capture recipe components that have “medication” identifiers (e.g., National Drug Codes) only, which practically excludes all components other than medication containers (e.g., vials) and fluid reservoirs (e.g., saline bags). As a result, any device components intrinsic to the final preparation (e.g., delivery devices, tubing, administration needles, etc.) or those used during a preparation (e.g., syringes, transfer needles, transfer devices, dispensing pins, filters, labels, etc.) are not included in this ingredient scanning step. If the same functionality is desired for devices, facilities must generate their own workarounds (e.g., applying “fake” barcodes to each device) to enforce preparation guidance and safety checks on device components (i.e., ensure the correct/compatible components are used, or detect/prevent incorrect or incompatible components from being used).

Further, even if non-medication components could be included, EHR guidance is limited to the “recipe” for the sterile preparation only and does not address required use steps, which are instead left latent or implied. Examples include but are not limited to using a particular preparation technique (e.g., removing air from a product reservoir, priming tubing), configuring a delivery device (e.g., programming a medication pump), affixing and positioning appropriate labels (e.g., to communicate required storage or administration conditions), and performing manipulations in a specific order (e.g., for patient safety or drug stability). In the context of multi-medication regimens, such as those common in oncology practice, several medications may have special conditions that require these types of unique use steps to ensure safe preparation and administration of high-alert medications.

Lack of adequate instruction on proper use steps is exacerbated further when the preparation involves use of a delivery device other than the hospital standard (i.e., conventional volumetric pumps). Due to their relative infrequency, such devices may be unfamiliar to most staff members, and improper device selection and use can have dire consequences. A current example is the preparation of fluorouracil into elastomeric pumps, which vary in their volume capacities and designated flow rates. As a result, the correct device must be selected from a range of options and filled correctly to ensure safe administration. Without mechanisms to enforce correct device selection and use, medication errors have been notoriously common with these systems, some of which have resulted in patient death.

As drug delivery device innovation continues to expand, novel devices with new and unfamiliar use models will likely become increasingly common in healthcare environments. These devices may have more complexity in terms of component selection, use steps, and other unique aspects such as designation of administration sequence for multi-medication regimens. As an example, new devices may employ technologies such as RFID that are sensitive to specific positioning or orientation, and reliance on HCP memory or past training alone to use such devices properly may be insufficient and potentially result in direct patient harm. Thus, improved solutions are needed to facilitate correct selection, use of, and verification of device components during sterile preparation, ideally embedding this capability into existing EHR systems and pharmacy workflows.

### SUMMARY

The present invention relates to improving medication preparation guidance and safety through expanded EHR functionality. More particularly, the invention relates to a method of instructing, facilitating, and verifying correct medication preparation, incorporating medication, device, and label components.

Thus, in a first aspect, the present invention provides a method of providing instructions for preparing or verifying the correct preparation of a medication for administration to a patient, the method comprising: (a) receiving, at a system, an indication of the medication to be prepared or that has been prepared for administration to the patient, (b) displaying, on a user interface of the system, a list of one or more items required for medication preparation, including medication, device, and label components, (c) scanning, at a scan-enabled client module of the system, a scannable code on each of the one or more items, (d) for at least one item of the one or more items, displaying, on the user interface of the system, a set of one or more instructions specific to at least one item of the one or more items, and (e) after assembling the one or more items required for medication preparation into an assembled unit, verifying one or more features of the assembled unit.

In one embodiment of the first aspect, the list of one or more items for preparing the medication are displayed in a sequential order, and the one or more items are scanned at the scan-enabled client module in the sequential order.

In another embodiment of the first aspect, the scannable code comprises one of a barcode, a quick response (QR) code, or a radio frequency identification (RFID).

In another embodiment of the first aspect, the set of one or more instructions specific to the at least one item of the one or more items are displayed as a pop-up in one of a sidebar, an additional tab, or a separate window of the user interface.

In another embodiment of the first aspect, the method further comprises displaying, on the user interface of the system, a window requesting a user input to confirm that the set of one or more instructions specific to the at least one item of the one or more items have been read.

In another embodiment of the first aspect, the method further comprises displaying, on the user interface of the system, a window requesting a user input to confirm that the set of one or more instructions specific to the at least one item of the one or more items have been completed.

In another embodiment of the first aspect, a next item of the one or more items cannot be scanned at the scan-enabled client module until the user input is received.

In another embodiment of the first aspect, a next item of the one or more items cannot be scanned at the scan-enabled client module until a threshold amount of time has elapsed after displaying the set of one or more instructions specific to the at least one item of the one or more items.

In another embodiment of the first aspect, the set of one or more instructions specific to the at least one item of the one or more items are displayed as a numbered list on the user interface.

In another embodiment of the first aspect, the set of one or more instructions specific to the at least one item of the one or more items are displayed as a video on the user interface.

In a second aspect, the present disclosure provides a system for providing instructions for preparing or verifying the correct preparation of a medication for administration to a patient, the system comprising: (a) a user interface, (b) a scan-enabled client module, (c) at least one memory storage element including data associated with the medication and/or the patient, (d) at least one processor, and (e) data storage including program instructions stored thereon that when executed by the at least one processor, cause the system to: (i) receive an indication of the medication to be prepared for administration to the patient, (ii) display, on the user interface, a list of one or more items for preparing the medication for administration to the patient, (iii) scan, at the scan-enabled client module, a scannable code on each of the one or more items; (iv) for at least one item of the one or more items, display, on the user interface, a set of one or more instructions specific to the at least one item of the one or more items, and (v) after assembling the one or more items required for medication preparation into an assembled unit, display, on the user interface, a request to verify one or more features of the assembled unit.

In one embodiment of the second aspect, the list of one or more items for preparing the medication are displayed in a sequential order, and wherein the one or more items are scanned at the scan-enabled client module in the sequential order.

In another embodiment of the second aspect, the system is further configured to display, on the user interface, a window requesting a user input to confirm that the set of one or more instructions specific to the at least one item of the one or more items have been read, wherein a next item of the one or more items cannot be scanned at the scan-enabled client module until the user input is received.

In another embodiment of the second aspect, the system is further configured to display, on the user interface, a window requesting a user input to confirm that the set of one or more instructions specific to the at least one item of the one or more items have been completed, wherein a next item of the one or more items cannot be scanned at the scan-enabled client module until the user input is received.

In another embodiment of the second aspect, a next item of the one or more items cannot be scanned at the scan-enabled client module until a threshold amount of time has elapsed after displaying the set of one or more instructions specific to the at least one item of the one or more items.

In another embodiment of the second aspect, the set of one or more instructions specific to the at least one item of the one or more items are displayed as a numbered list on the user interface.

In another embodiment of the second aspect, the set of one or more instructions specific to the at least one item of the one or more items are displayed as a video on the user interface.

In another embodiment of the second aspect, the set of one or more instructions comprise one or more of a component selection instruction, a component unpackaging instruction, a component handling instruction, a component assembly instruction, a component dispensing instruction, a component storage instruction, an administration instruction, a disposal instruction, a recycling instruction, a labeling instruction, a programming instruction or a packaging instruction.

In another embodiment of the second aspect, the set of one or more instructions are expressed as a positive or a negative instruction.

In another embodiment of the second aspect, the request to verify one or more features of the assembled unit directs a user to scan one or more components of the assembled unit to verify if the set of one or more instructions have been completed correctly.

In another embodiment of the second aspect, the request to verify one or more features of the assembled unit directs a user to scan one or more components of the assembled unit to verify one or more of a physical presence, a physical characteristic, a physical location, a visual readability, a machine readability, information contained on/in RFID tag or NFC tag, or combinations thereof.

In another embodiment of the second aspect, the verification step is performed in comparison to expected information contained in a medication order.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

FIG. 1 illustrates a simplified block diagram of a system for providing instructions for preparing a medication for administration to a patient, according to an example embodiment.

FIG. 2 illustrates a screen shot of the user interface of the system of FIG. 1, according to an example embodiment.

FIG. 3 is a block diagram of a method of providing instructions for preparing and verifying the preparation of a medication that is to be administered to a patient using a drug delivery system based upon a medication order, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

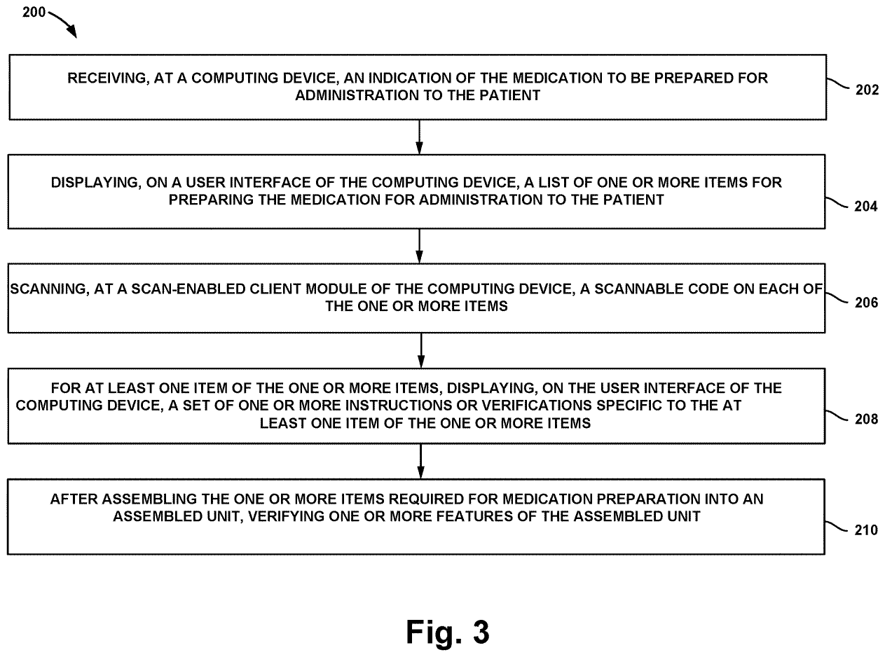

In FIG. 3, referred to above, the blocks may represent operations and/or portions thereof and lines connecting the various blocks do not imply any particular order or dependency of the operations or portions thereof. It will be understood that not all dependencies among the various disclosed operations are necessarily represented. FIG. 3 and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

The present invention provides systems and methods for providing instructions for preparing or verifying a medication for administration to a patient. In particular, when scanning a scannable item (such as barcode, QR code or in the future RFID) on a medication, medication package, patient-specific medication label, auxiliary medication label, label that indicates medication delivery instructions or sequencing instruction, device, device component (e.g., tubing set), or device package that includes the product identifier to complete the “recipe” of a sterile preparation for the medication for administration, the scannable item could lead the electronic health record (EHR) system to provide instructions for the preparation/use in the sidebar, another tab, or separate window on the user interface of the system. This may be the same scannable item as the product identifier or a separate one. The instructions may be specific to device components, labels or other ancillary components, or be customized to include information for the medication(s) being prepared, particularly in the context of multi-medication regimens and medication sequencing. As an example, instruction may be specific to correct placement of medication labels or other labels, with or without embedded RFID, NFC, or other technology, and include guidance on correct placement of such labels, such as position and/or orientation relative to device components or other labels. Additionally, instruction may be specific to direct the user to avoid placement of such labels or other adhesives (e.g., port seals) in areas which may impact device function. As additional examples, instruction may also be specific to either disassembly or assembly of device components to ensure proper device function or specific to supplied device packaging, re-use of the supplied packaging, or co-packaging of one or more of each device and medication components.

Scanning an item would cause instructions to correctly prepare/use the components in the medication preparation, including any devices, to appear in the EHR or another webpage in some form. This would allow for easy access of instructions and could even be a required step to “complete” the preparation in the EHR to ensure that the preparation/use instructions are read and in particular, that any device components are being prepared/used correctly. This removes the burden of accessing other forms of instructions and removes the risk of not using any form of them. Further, as HCPs are required to scan device components as a part of the “recipe” along with medication components, no additional user steps are required. A similar scanning step would be used to verify that the preparation was completed correctly, such as correct assembly of device components, correct packaging, presence of required labels, or correct positioning of such labels relative to device components or other labels.

By giving HCPs seamless access to preparation and verification instructions, health systems can reduce risk of preparation errors and ensure a higher level of patient safety. HCP confidence in correct preparation will also be increased, potentially reducing workflow disruption and improving efficiency.

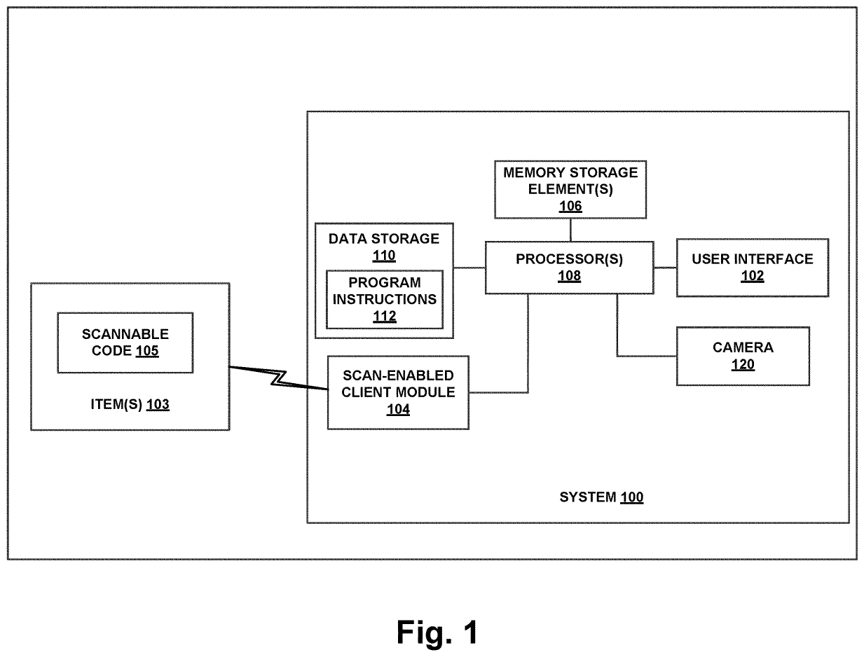

With reference to the Figures, FIG. 1 illustrates a system 100 for providing instructions for preparing a medication of the correct preparation of a medication for administration to a patient. In particular, as shown in FIG. 1, the system 100 includes a user interface 102. The user interface 102 may be, for example, an optical see-through display, an optical see-around display, or a video see-through display, as non-limiting examples. The system 100 further includes a scan-enabled client module 104. The scan-enabled client module 104 may include scannable code reader module configured to read a scannable code as discussed in additional detail below. In one exemplary embodiment, the scan-enabled client module 104 may include a native application that is adapted to execute on the system 100, and in such a case that native application may include scanning/decoding capability or alternatively scan-enabled client module 104 may simply invoke the services of a third-party scanner/decoder that is installed in the system 100. Other implementations of the scan-enabled client module 104 are possible as well.

The system 100 further includes at least one memory storage element 106 including data associated with the medication and/or the patient. In particular, the at least one memory storage element 106 may include data associated with a plurality of instructions for preparing a plurality of medications that a plurality of medications were prepared correctly. The at least one memory storage element 106 can include any type of memory now known or later developed including but not limited to volatile memory (such as RAM), non-volatile memory (such as ROM, flash memory, etc.) or any combination thereof.

The system 100 further includes at least one processor 108 and data storage 110 including program instructions 112 stored thereon that when executed by the at least one processor 108, cause the system 100 to perform functions. Although various components of the system 100 are shown as distributed components, it should be understood that any of such components may be physically integrated and/or distributed according to the desired configuration of the system. Depending on the desired configuration, the at least one processor 108 can be any type of processor including, but not limited to, a microprocessor, a microcontroller, a digital signal processor, or any combination thereof. In particular, the functions include (i) receive an indication of the medication to be administered to the patient, (ii) display, on the user interface 102, a list of one or more items 103 for administering the medication to the patient, (iii) scan, at the scan-enabled client module 104, a scannable code 105 on each of the one or more items 103, (iv) for at least one item of the one or more items, display, on the user interface 102, a set of one or more instructions specific to the at least one item of the one or more items, and (v) after assembling the one or more items required for medication preparation into an assembled unit, display, on the user interface (102), a request to verify one or more features of the assembled unit.

In one example, the scannable code 105 comprises one of a barcode, a quick response (QR) code, or a radio frequency identification (RFID).

In one example, the list of one or more items for administering the medication are displayed in a sequential order on the user interface 102, and the one or more items 103 are scanned at the scan-enabled client module 104 in the sequential order.

In one example, the set of one or more instructions specific to the at least one item of the one or more items are displayed as a pop-up in one of a sidebar, an additional tab, or a separate window of the user interface.

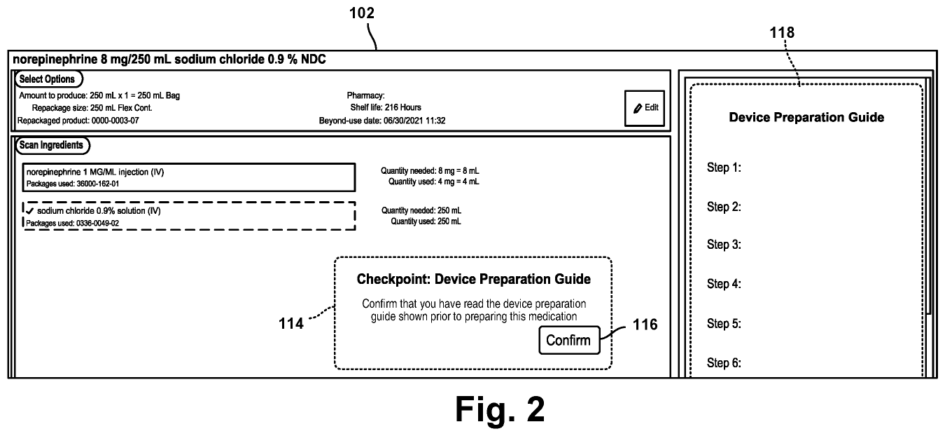

FIG. 2 illustrates a screen shot of the user interface 102 of the system 100. As shown in FIG. 2, in one example the system 100 is further configured to display, on the user interface 102, a window 114 requesting a user input 116 to confirm that the set of one or more instructions 118 specific to the at least one item of the one or more items have been read. In one such example, a next item of the one or more items cannot be scanned at the scan-enabled client module 104 until the user input is received. In one such example, the scan-enabled client module 104 is disabled or turned off until the user input is received indicating that the set of one or more instructions 118 specific to the at least one item of the one or more items have been read.

In another example, the system 100 is further configured to display, on the user interface 102, a window 114 requesting a user input 116 to confirm that the set of one or more instructions 118 specific to the at least one item of the one or more items have been completed. In one such example, a next item of the one or more items cannot be scanned at the scan-enabled client module 104 until the user input is received. In one such example, the scan-enabled client module 104 is disabled or turned off until the user input is received indicating that the set of one or more instructions 118 specific to the at least one item of the one or more items have been completed.

In another example, a next item of the one or more items cannot be scanned at the scan-enabled client module until a threshold amount of time has elapsed after displaying the set of one or more instructions specific to the at least one item of the one or more items. In one such example, the scan-enabled client module 104 is disabled or turned off until the threshold amount of time has elapsed indicating that the set of one or more instructions 118 specific to the at least one item of the one or more items have been read. The threshold amount of time may be 10 seconds, 20 seconds, 30 seconds, 40 seconds, 50 seconds, or 60 seconds as non-limiting examples. The threshold amount of time may vary based on the set of one or more instructions. In one example, as shown in FIG. 2, the set of one or more instructions specific to the at least one item of the one or more items are displayed as a numbered list on the user interface 102. In one example, the numbered list may further include one or more static figures providing further clarification of the set of one or more instructions.

In another example, the set of one or more instructions specific to the at least one item of the one or more items are displayed as a video on the user interface 102. The video may comprise an instructional video that walks the HCP through the necessary steps.

In another example, the system 100 further includes a camera 120. The camera 120 may be configured to take a picture and/or video of each step of the preparation of the medication. The pictures and/or videos may then be stored in the data storage 110 for future review.

In one example, the set of one or more instructions comprise one or more of a component selection instruction, a component unpackaging instruction, a component handling instruction, a component assembly instruction, a component dispensing instruction, a component storage instruction, an administration instruction, a disposal instruction, a recycling instruction, a labeling instruction, a programming instruction or a packaging instruction. The set of one or more instructions may be expressed as a positive (e.g., “drug is compatible,” etc.) or negative (e.g., “drug is not compatible,” “do not use,” “contraindication,” etc.).

In another example, the request to verify one or more features of the assembled unit directs a user to scan one or more components of the assembled unit to verify if the set of one or more instructions have been completed correctly.

In another example, the request to verify one or more features of the assembled unit directs a user to scan one or more components of the assembled unit to verify one or more of a physical presence of the one or more components of the assembled unit, a physical characteristic of the one or more components of the assembled unit, a physical location of the one or more components of the assembled unit, a visual readability of the one or more components of the assembled unit, a machine readability of the one or more components of the assembled unit, information contained on/in RFID tag or NFC tag of the one or more components of the assembled unit, or combinations thereof.

FIG. 3 is a block diagram of an example method of providing instructions for preparing and verifying the preparation of a medication that is to be administered to a patient using a drug delivery system based upon a medication order. Method 200 shown in FIG. 3 presents an embodiment of a method that could be used by the system 100 as described in FIGS. 1-2, as examples. Method 200 may include one or more operations, functions, or actions as illustrated by one or more of blocks 202-210. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 200 and other processes and methods disclosed herein, the block diagram shows functionality and operation of one possible implementation of present embodiments. In this regard, each block may represent a module, a segment, or a portion of program code, which includes one or more instructions executable by a processor or system for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

Initially, at block 202, the method 200 includes receiving, at a system, an indication of the medication to be administered to the patient. At block 204, the method 200 includes displaying, on a user interface of the system, a list of one or more items for preparing the medication for administration to the patient. At block 206, the method 200 includes scanning, at a scan-enabled client module of the system, a scannable code on each of the one or more items. At block 208, the method 200 further includes, for at least one item of the one or more items, displaying, on the user interface of the system, a set of one or more instructions s specific to the at least one item of the one or more items. At block 210, the method 200 further includes after assembling the one or more items required for medication preparation into an assembled unit, verifying one or more features of the assembled unit.

In one example, the list of one or more items for administering the medication are displayed in a sequential order, and the one or more items are scanned at the scan-enabled client module 104 in the sequential order.

In one example, the scannable code comprises one of a barcode, a quick response (QR) code, or a radio frequency identification (RFID).

In one example, the set of one or more instructions specific to the at least one item of the one or more items are displayed as a pop-up in one of a sidebar, an additional tab, or a separate window of the user interface.

In one example, the method 200 further includes, displaying, on the user interface of the system, a window requesting a user input to confirm that the set of one or more instructions specific to the at least one item of the one or more items have been read. In one such example, a next item of the one or more items cannot be scanned at the scan-enabled client module until the user input is received.

In one example, the method 200 further includes, displaying, on the user interface of the system, a window requesting a user input to confirm that the set of one or more instructions specific to the at least one item of the one or more items have been completed. In one such example, a next item of the one or more items cannot be scanned at the scan-enabled client module until the user input is received.

In one example, a next item of the one or more items cannot be scanned at the scan-enabled client module until a threshold amount of time has elapsed after displaying the set of one or more instructions specific to the at least one item of the one or more items.

In one example, the set of one or more instructions specific to the at least one item of the one or more items are displayed as a numbered list on the user interface. In another example, the set of one or more instructions specific to the at least one item of the one or more items are displayed as a video on the user interface.

In one example, the set of one or more instructions comprise one or more of a component selection instruction, a component unpackaging instruction, a component handling instruction, a component assembly instruction, a component dispensing instruction, a component storage instruction, an administration instruction, a disposal instruction, a recycling instruction, a labeling instruction, a programming instruction or a packaging instruction. The set of one or more instructions may be expressed as a positive (e.g., “drug is compatible,” etc.) or negative (e.g., “drug is not compatible,” “do not use,” “contraindication,” etc.).

In another example, verifying one or more features of the assembled unit comprises a user scanning one or more components of the assembled unit to verify if the set of one or more instructions have been completed correctly.

In another example, verifying one or more features of the assembled unit comprises a user scanning one or more components of the assembled unit to verify one or more of a physical presence of the one or more components of the assembled unit, a physical characteristic of the one or more components of the assembled unit, a physical location of the one or more components of the assembled unit, a visual readability of the one or more components of the assembled unit, a machine readability of the one or more components of the assembled unit, information contained on/in RFID tag or NFC tag of the one or more components of the assembled unit, or combinations thereof.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation W137 (CDW137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7 +3, 5 +2, 7 +4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

1-22. (canceled)

23. A method of providing instructions for preparing and verifying the preparation of a medication that is to be administered to a patient using a drug delivery system based upon a medication order, the method comprising:
receiving, at a system, an indication of the medication to be prepared for administration to the patient;
displaying, on a user interface of the system, a list of one or more items required for medication preparation, including medication, device, and label components;
scanning, at a scan-enabled client module of the system, a scannable code on each of the one or more items;
for at least one item of the one or more items, displaying, on the user interface of the system, a set of one or more instructions specific to the at least one item of the one or more items; and
after assembling the one or more items required for medication preparation into an assembled unit, verifying one or more features of the assembled unit.

24. The method of claim 23, wherein the list of one or more items for preparing the medication are displayed in a sequential order, and wherein the one or more items are scanned at the scan-enabled client module in the sequential order.

25. The method of claim 23, wherein the scannable code comprises one of a barcode, a quick response (QR) code, or a radio frequency identification (RFID).

26. The method of claim 23, wherein the set of one or more instructions specific to the at least one item of the one or more items are displayed as a pop-up in one of a sidebar, an additional tab, or a separate window of the user interface.

27. The method of claim 23, further comprising:
displaying, on the user interface of the system, a window requesting a user input to confirm that the set of one or more instructions specific to the at least one item of the one or more items have been read.

28. The method of claim 23, further comprising:
displaying, on the user interface of the system, a window requesting a user input to confirm that the set of one or more instructions specific to the at least one item of the one or more items have been completed.

29. The method of 27, wherein a next item of the one or more items cannot be scanned at the scan-enabled client module until the user input is received.

30. The method of claim 23, wherein a next item of the one or more items cannot be scanned at the scan-enabled client module until a threshold amount of time has elapsed after displaying the set of one or more instructions specific to the at least one item of the one or more items.

31. The method of claim 23, wherein the set of one or more instructions specific to the at least one item of the one or more items are displayed as a numbered list on the user interface.

32. The method of claim 23, wherein the set of one or more instructions specific to the at least one item of the one or more items are displayed as a video on the user interface.

33. A system for providing instructions for preparing a medication for administration to a patient, the system comprising:
a user interface;
a scan-enabled client module;
at least one memory storage element including data associated with the medication and/or the patient;
at least one processor; and
data storage including program instructions stored thereon that when executed by the at least one processor, cause the system to:
receive an indication of the medication to be prepared for administration to the patient;
display, on the user interface, a list of one or more items for preparing the medication for administration to the patient;
scan, at the scan-enabled client module, a scannable code on each of the one or more items;
for at least one item of the one or more items, display, on the user interface, a set of one or more instructions specific to the at least one item of the one or more items; and
after assembling the one or more items required for medication preparation into an assembled unit, display, on the user interface, a request to verify one or more features of the assembled unit.

34. The system of claim 33, wherein the list of one or more items for preparing the medication are displayed in a sequential order, and wherein the one or more items are scanned at the scan-enabled client module in the sequential order.

35. The system of claim 33, wherein the system is further configured to:
display, on the user interface, a window requesting a user input to confirm that the set of one or more instructions specific to the at least one item of the one or more items have been read, wherein a next item of the one or more items cannot be scanned at the scan-enabled client module until the user input is received.

36. The system of claim 33, wherein the system is further configured to:
display, on the user interface, a window requesting a user input to confirm that the set of one or more instructions specific to the at least one item of the one or more items have been completed, wherein a next item of the one or more items cannot be scanned at the scan-enabled client module until the user input is received.

37. The system of claim 33, wherein a next item of the one or more items cannot be scanned at the scan-enabled client module until a threshold amount of time has elapsed after displaying the set of one or more instructions specific to the at least one item of the one or more items.

38. The system of claim 33, wherein the set of one or more instructions specific to the at least one item of the one or more items are displayed as a numbered list on the user interface.

39. The system of claim 33, wherein the set of one or more instructions specific to the at least one item of the one or more items are displayed as a video on the user interface.

40. The system of claim 33, wherein the set of one or more instructions comprise one or more of a component selection instruction, a component unpackaging instruction, a component handling instruction, a component assembly instruction, a component dispensing instruction, a component storage instruction, an administration instruction, a disposal instruction, a recycling instruction, a labeling instruction, a programming instruction or a packaging instruction.

41. The system of claim 33, wherein the set of one or more instructions are expressed as a positive or a negative instruction.

42. The system of claim 33, wherein the request to verify one or more features of the assembled unit directs a user to scan one or more components of the assembled unit to verify if the set of one or more instructions have been completed correctly.

43. The system of claim 33, wherein the request to verify one or more features of the assembled unit directs a user to scan one or more components of the assembled unit to verify one or more of a physical presence, a physical characteristic, a physical location, a visual readability, a machine readability, information contained on/in RFID tag or NFC tag, or combinations thereof.

44. The system of claim 33, wherein the verification step is performed in comparison to expected information contained in a medication order.
